(19) United States
(12) Patent Application Publication
(10) Pub. No.: US 2022/0133195 A1
(43) Pub. Date: May 5, 2022

(54) APPARATUS, SYSTEM, AND METHOD FOR ASSESSING AND TREATING EYE CONTACT AVERSION AND IMPAIRED GAZE

(71) Applicant: Click Therapeutics, Inc., New York, NY (US)

(72) Inventor: Brian Iacoviello, New York, NY (US)

(73) Assignee: Click Therapeutics, Inc., New York, NY (US)

(21) Appl. No.: 17/518,557

(22) Filed: Nov. 3, 2021

Related U.S. Application Data
(60) Provisional application No. 63/108,872, filed on Nov. 3, 2020.

(51) Int. Cl. A61B 5/16; G16H 50/30; G16H 20/70

(52) U.S. Cl. CPC A61B 5/163; G16H 20/70; G16H 50/30

(57) ABSTRACT

Provided for may be a system for assessing an eye contact aversion bias comprising initiating a first predetermined number of trials, wherein a trial comprises generating a displayed human face having a first spatial location corresponding to a first feature and a second spatial location corresponding to a second feature, wherein the first feature is the eyes of the displayed human face, and the second feature is not the eyes of the displayed human face, generating a target stimulus, wherein the target stimulus is presented at the first or the second spatial location, and receiving a selection signal. The system may further comprise calculating a response time, a first average response time, a second average response time, and calculating the eye contact aversion bias.

### CLAIM OF PRIORITY

This application claims priority from U.S. Provisional Patent Application No. 63/108,872, filed on Nov. 3, 2020, the contents of which are incorporated herein by reference.

### FIELD OF THE INVENTION

Embodiments of the invention relate generally to an apparatus, system, and method for assessing and treating individuals with eye contact aversion and impaired gaze maintenance.

### INTRODUCTION

Eye contact aversion and impaired gaze maintenance are often indicators of various mental diseases, emotional diseases, and other health conditions including depression disorders, bipolar disorder, anxiety disorders such as social anxiety disorder, as well as various developmental disorders such as autism. Undiagnosed or improperly treated psychiatric conditions carry a substantial risk to an individual, including difficulty while learning, social ostracization, and even self-harm. Impaired gaze maintenance and poor eye contact are common symptoms of autism disorder, schizophrenia, depression, brain trauma, and bipolar disorder. That is, irregular eye movement is often an indication of defects in neural circuitry.

More specifically, for individuals with autism, maintaining eye contact with another person may be extremely difficult. Further, an individual diagnosed with schizophrenia may exhibit an inability to focus on slow-moving objects. As another example, individuals suffering from brain trauma may experience extreme ocular motility dysfunction when attempting to visually focus on different targets.

Additionally, impaired gaze maintenance or decreased eye mobility may be an indication of drug or alcohol use. For example, individuals under the influence of alcohol may experience horizontal gaze nystagmus, a condition where the eye struggles to focus on horizontally moving objects. Consequently, officers often conduct field sobriety tests to detect impaired gaze. However, these tests are quite subjective and could be greatly improved with an apparatus or method that more empirically and objectively detects impaired gaze.

Prediction of developing or undiagnosed mental conditions may involve substantial guesswork. While there are clear relationships between impaired eye functionality and mental conditions, determining and quantifying such a relationship, and predicting the severity of the impairment for a particular individual, has proven elusive and difficult.

It would be desirable, therefore, to provide apparatuses, systems, and methods for detecting and quantifying impaired eye contact and poor gaze maintenance.

It would be further desirable to provide apparatuses, systems, and methods for alerting individuals to an increased likelihood of health conditions associated with impaired eye function, and providing remediation opportunities to such individuals.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 illustrates a block diagram of a distributed computer system that can implement one or more aspects of an embodiment of the present invention.

FIG. 2 illustrates a block diagram of an electronic device that can implement one or more aspects of an embodiment of the invention.

FIG. 3 shows an embodiment of a workflow depicting the generated screens of a trial.

FIG. 4 shows an embodiment of a workflow depicting the generated screens of a trial.

FIG. 5 shows an embodiment of a training phase.

FIG. 6 is a workflow depicting an embodiment of a training phase method.

### DETAILED DESCRIPTION

For this disclosure, singular words should be construed to include their plural meaning, unless explicitly stated otherwise. Additionally, the term “including” is not limiting. Further, “or” is equivalent to “and/or,” unless explicitly stated otherwise. Although, ranges may be stated as preferred, unless stated explicitly, there may exist embodiments that operate outside of preferred ranges.

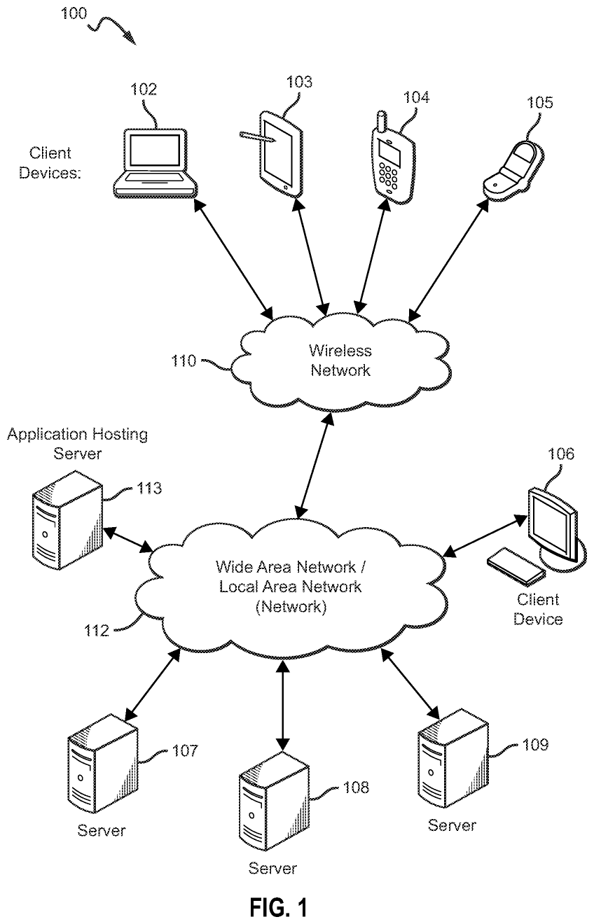

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

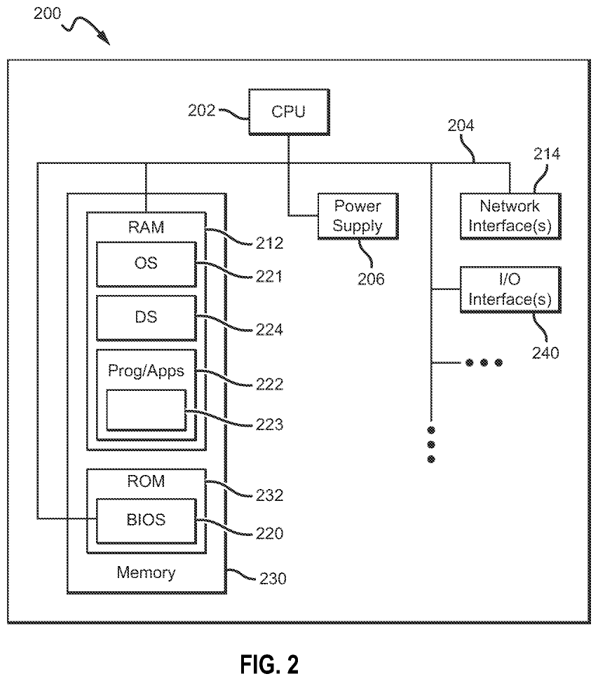

FIG. 2 illustrates a block diagram of an electronic device 200 that can implement one or more aspects of an apparatus, system and method for increasing mobile application user engagement (the “Engine”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components, and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a pl1urality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

As noted above, embodiments of the present invention relate to apparatuses, methods and systems of detecting/determining impaired eye function and the subsequent likelihood of health conditions via presentation and assessment of visual stimuli running on an electronic device. The embodiments may be referred to as the impaired eye function assessment (IEFA) system, or simply the “system.”

In one embodiment, the IEFA system may include software running on a smartphone of a user, an IEFA apparatus.

The IEFA system, may present one or more photographs and/or videos, for example, of a person's face, to make certain predictions regarding the user's likelihood of impaired eye function, as discussed in more detail below.

In one embodiment, the IEFA system may comprise two phases, an assessment phase and a training phase.

In one embodiment, an IEFA system may comprise a predetermined number of trials during which an image of a displayed human face is presented to the user, followed by a target stimulus presented in a spatial location corresponding to a location at or near the displayed human face. For example, in some trials the target stimulus is presented at or near the eyes, while in other trials the target stimulus is presented at another spatial location. In some embodiments, trials may comprise starting screens, inter-stimulus screens, and/or evaluation screens.

In an embodiment, the target stimulus may be the letter “E” and/or “F.” In other embodiments, the target stimulus may be a symbol, icon, or the like, such as a dot probe or a fixation cross. In an embodiment, the target stimulus screen is a solid black blank screen where the target stimulus is disposed in front. In further embodiments, the target stimulus screen may be any solid color. In alternative embodiments, the target stimulus screen may display an image, photograph, or video. An evaluation screen may follow the target stimulus screen. The evaluation screen may present the user with a question asking the user which target stimulus they previously observed or the location of the target stimulus. For example, in one embodiment, the evaluation screen questions the user whether the user observed an “E” or “F” (or any potentially presented target stimuli).

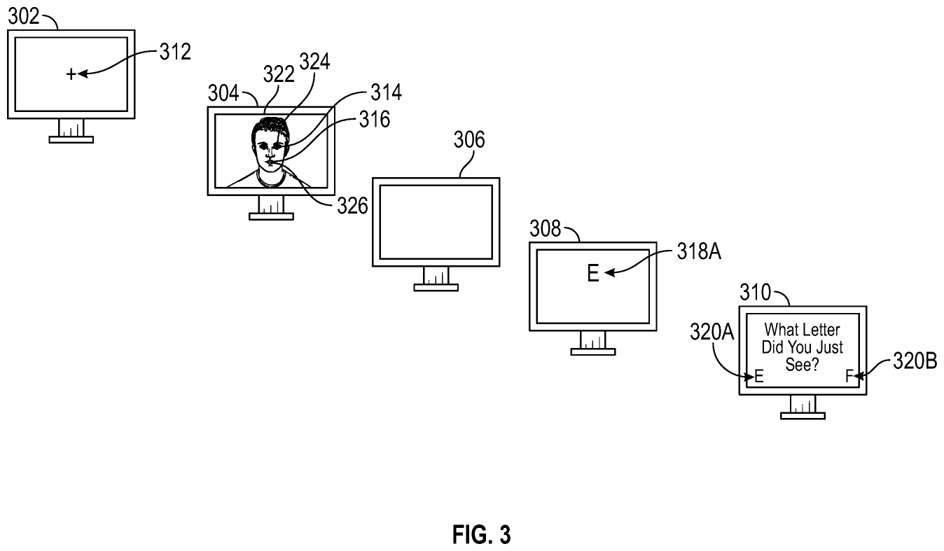

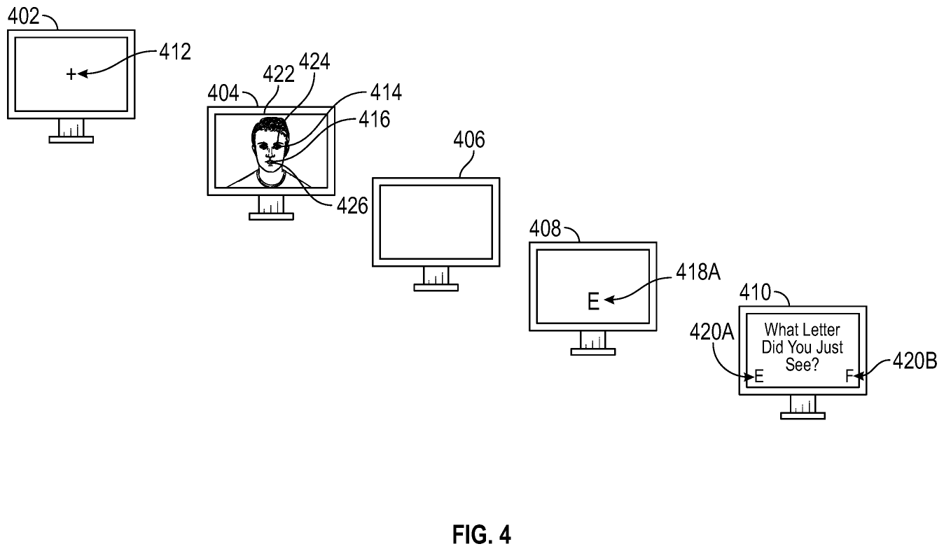

Referring to FIGS. 3-4, the IEFA system may display a number of screens to the user via the electronic device. In one embodiment, the user is presented with a starting screen 302, a facial stimulus screen 304, a blank screen 306, a target stimulus screen 308, and an evaluation screen 310. In alternative embodiments, any number of screens may be displayed in any combination or order. For the purpose of this disclosure, “screen” may refer to a graphical user interface presented on an electronic device.

In an embodiment, the user is presented with a starting screen 302, where the starting screen 302 is a blank black screen with an indicator 312 disposed on the center of the screen. The indicator 312 may be disposed at an indicator spatial location. The starting screen 302 may be any solid color. In alternate embodiments, the starting screen 302 may be any photo, image, or video. In one embodiment, the indicator 312 is a white “fixation cross.” However, in alternate embodiments, the indicator 312 may be any symbol, for example, an “x,” a dot, a circle, any crosshair, any reticle, or other image. Additionally, the indicator 312 may be any color. In alternate embodiments, the indicator 312 may flash, strobe, or change opacity or color.

In various embodiments, the indicator 312 may be located at any spatial position on the starting screen 302. In one embodiment, the starting screen 302 is presented to the user for 50 to 1000 milliseconds before disappearing. In another embodiment, the starting screen 302 is presented for 500 to 1000 milliseconds. However, there exist alternate embodiments where the starting screen 302 is displayed for less than 50 milliseconds or more than 1000 milliseconds.

In an embodiment, the facial stimulus screen 304 follows the starting screen 302. The facial stimulus screen 304 may display an image of a face, a displayed human face 322. However, in alternate embodiments the facial stimulus screen 304 may display other images, including, but not limited to, the face of an animal, an excerpt of text, or a work of art. In such embodiments, the calculated response times, average response times, and attention biases may be based on the user's visual focus as it related to certain shapes, colors, or words.

The displayed human face 322 may express any number of emotions, including, but not limited, to happiness, sadness, surprise, fear, apathy, anger, or disappointment. Further, the displayed human face 322 may be any age, gender, race, or other identifying characteristic. In one embodiment, the displayed human face 322 is a static image. In another embodiment, the displayed human face 322 is a video depicting the human face in motion. In further embodiments, the displayed human face 322 may be a video of the displayed human changing their facial expressions and/or emotions. The facial stimulus screen 304 may depict the displayed human face 322 on a blank background. This blank background may be a solid color (such as black or white) or may be any image, photo, or video. In one embodiment, the facial stimulus screen 304 is displayed to the user for 20 to 2000 milliseconds before disappearing. However, the facial stimulus screen 322 may be displayed for any suitable period of time.

Any number of the screens may include a first spatial location 314 and a second spatial location 316. For example, the first spatial location 314 may correspond to a first feature 324 and the second spatial location 316 may correspond to a second feature 326. The first feature 324 and the second feature 326 may be any aspect of the image displayed on the facial stimulus screen 304. For example, the first feature 324 may be the eyes of the displayed human face 322 and the second feature 326 may be the mouth of the displayed human face 322. The first spatial location 314 and the second spatial location 316 may be a fixed coordinate, consistent across any number of screens.

In an embodiment, the blank screen 306 follows the facial stimulus screen 304. The blank screen 306 may be a blank screen of a solid color. However, there are alternate embodiments where the blank screen 306 is any image, photograph, or video. The blank screen 306 may appear for a duration of at least 500 milliseconds. However, in other embodiments the blank screen 306 may appear for less than 500 milliseconds. The duration of the blank screen 306 may be referred to as the inter-stimulus interval (ISI).

In an embodiment, the target stimulus screen 308 follows the blank screen 306. The target stimulus screen 308 may share the same background as the blank screen 306. However, the target stimulus screen 308 background may be any solid color or any image, photograph, or video. A target stimulus 318 may be disposed on the target stimulus screen 308. The target stimulus 318 may be any symbol, icon, or the like, such as an “x,” a fixation cross, a probe, a dot, a circle, any crosshair, any reticle, or other image. The target stimulus 318 may be one of two similar-looking symbols associated with keyboard keys. As a non-limiting example, the target stimulus 318 may be one of the following pairs of letters: “E” or “F”, “p” or “b,” or “b” or “d.” For example, target stimulus 318A may be “E” and target stimulus 318B may be “F.” In alternate embodiments, the target stimulus 318 may be one of three similar-looking symbols. In further embodiments, the target stimulus 318 may be one of any number of similar-looking symbols. In other embodiments, the number of similar-looking symbols presented to the user are determined based on at least one of: demographic information regarding the user, results of evaluation step in prior uses (trials) of the IEFA system by the user, results of evaluation step of the IEFA system by other users in the user's demographic.

In some trials, the target stimulus 318 may be positioned at the same spatial location as the spatial location of the displayed human face's eyes on the facial stimulus screen 304. In other trials, the target stimulus 318 may be positioned at the same spatial location as the spatial location of the displayed human face's mouth on the facial stimulus screen 304. In other trials, the target stimulus 318 may be positioned at any spatial location correlated to any one of the parts of the displayed human face 322 (or other image). In other trials, the target stimulus 318 may be positioned at a spatial location that was not occupied by the displayed human face 322. The target stimulus 318 and/or target stimulus screen 308 is displayed to the user for 20 to 2000 milliseconds before disappearing. The time period for which the target stimulus 318 and/or target stimulus screen 308 is displayed to the user may be determined based on at least one of: demographic information regarding the user, results of evaluation step in prior uses (trials) of the IEFA system by the user, results of evaluation step of the IEFA system by other users in the user's demographic.

In an embodiment, an evaluation screen 310 follows the target stimulus screen 308. The evaluation screen 310 may have a background that is the same as the background of the target stimulus screen 308, blank screen 306, facial stimulus screen 304, and/or starting screen 302. The evaluation screen 310 may have a background that is any image, photograph, or video. The evaluation screen 310 may display text to the user. The text may be a question to the user, asking the user the location of the target stimulus 381, or, in some embodiments, which target stimulus 318 the user observed. As a non-limiting example, the question may be: “what letter did you just see?” The user may be presented with an option to select between the two or more target stimuli 318 (for example, by using a touchscreen on a smart phone, pressing a key on a keyboard, or other suitable methods). In such an embodiment, the evaluation screen 310 may generate a first selectable target stimulus 320A and a second selectable target stimulus 320B. The evaluation screen 310 may display the first selectable target stimulus 320A and the second selectable target stimulus 320B (or any other target stimuli 318) to the user. However, in alternate embodiments, the evaluation screen 310 does not display the two or more potential target stimuli 318. Instead, in such an alternate embodiment, the user may type, draw (for example, with a finger or a stylus on a touchscreen), or otherwise indicate the location of, or the target stimuli 318 they previously observed, without being presented a list of options.

In one embodiment, the IEFA system may include a touchscreen, enabling the user to select the location of the target stimuli, or select which of the two or more target stimuli 318 the user believes they observed. However, in alternate embodiments, the IEFA system may include a series of buttons, a mouse, a track pad, or other means of allowing the user the make a selection. In another embodiment, the IEFA system includes a microphone that enables the user to make vocal confirmations and selections. In such an embodiment, the user may be able to answer the prompts of the evaluation screen 310 by vocalizing their selection.

In one embodiment, the IEFA system does not inform the user whether or not the user made the correct selection. Although, in an alternate embodiment the IEFA system does inform the user whether or not the user made the correct selection. The evaluation screen 310 may remain displayed to the user until the user makes a selection. In another embodiment, the evaluation screen 310 has a set duration, causing the evaluation screen 310 to disappear after a set time limit. In an embodiment, once the user makes a selection or the time limit has expired, the evaluation screen 310 disappears. In a further embodiment, the evaluation screen 310 presents the user with the option of choosing “neither” target stimuli 318 or recording that they “did not know” which target stimuli 318 was previously presented. Alternatively, a lack of answering or lapse of time (even if answered) may be recorded. In another further embodiment, the IEFA system may correlate uncertain answers or lapses of time to the response times, average response times, and/or attention biases of particular trials or sessions. As a non-limiting example, utilizing the foregoing method may illuminate that the user has increased difficulty when observing a particular kind of displayed human face 322 or part of the displayed human face 322.

In an embodiment, the user continues to the next trial where the user is once again presented with the starting screen 302. However, in various embodiments, any one of the screens may follow the evaluation screen 310. In an alternate embodiment, the IEFA system maintains a tally, record, or score that is displayed to the user. In one embodiment, the number of correct selections is represented as a tally, record, or score. The tally, record, or score, may be displayed to the user on any number of the screens.

In an embodiment, once the user has responded to the evaluation screen 310, a new trial begins. The new trial may present a target stimulus 318 in a spatial location that is the same or different than the spatial location of the previous trial. In an alternate embodiment, only the first trial of a session includes a starting screen 302, every subsequent trial generates the facial stimulus screen 304 after the evaluation screen 310.

In an embodiment, the IEFA system includes at least a memory and a processor. In one embodiment, the IEFA system records the response time and/or whether the user correctly identified the target stimulus 318. In an embodiment, the response time may be the time elapsed between the time the evaluation screen was displayed to the user and time when the user answered the evaluation screen's prompt. In one embodiment, the response time (RT) may be the difference between the time when the user answers the prompt on the evaluation screen 310 and the time when the target stimulus 318 is first presented to the user. The RT may be represented, in a non-limiting example, as: RT=T_{R}−T_{R}, where RT is the response time, T_{R} is the time of the user's response, and T_{P} is the time of the target stimulus presentation (for example, on the evaluation screen 310). As a non-limiting example, if the user responded to the target stimulus 318 at 11:23:05 AM and the selectable stimuli 320A/320B were presented at 11:23:04 AM, then the RT would be 1 second or 1000 milliseconds.

In an embodiment, the user may complete the assessment phase just before and/or just after the training phase. In further embodiments, the assessment phase is configured to determine the user's eye contact aversion bias. In an embodiment, the user's eye contact aversion bias may be calculated as the difference in the reaction time for target stimuli presented in the location of the displayed face's eyes and the reaction time when target stimuli 318 is presented in the other spatial location on the face.

In one embodiment, the memory contains a spread sheet or other record keeping means that records at least the RT, and the T_{R}, T_{P}, whether or not the user made the correct selection, and/or the number of trials and sessions that the user has performed. The number of trials of a particular session may be pre-determined. However, alternatively, there may be no limit to the number of trials in a particular session. In such an embodiment, the number of trials may be the number of trials performed by the user before the user abandons the IEFA system.

In one embodiment, the data is preprocessed. For example, the data may be transformed, or encoded, such that the system may easily parse it. Further, the data may be preprocessed such that the data may be easily interpreted by a machine learning algorithm. Data may be categorized and sorted into bins. The system may also correct data with missing, inconsistent, and/or duplicative values. In one embodiment, the correct trials set may be the set of data correlating to the trials where the user correctly answered the evaluation prompt. For example, each trial may have an associated selection status. The selection status may indicate whether the user correctly selected the location of the target stimuli, or selected the selectable target stimuli 320, at the evaluation screen 310. The incorrect trials may be the set of data correlating to the trials where the user incorrectly answered the evaluation prompt. In one embodiment, the incorrect trials are excluded from further calculations. In further embodiments, the incorrect trials may be used in a limited capacity, as a weight, modifier or have some other effect on either the correct trial data set or final calculations. In an embodiment, the processor determines which trials were correct and incorrect. However, there exist other embodiments where other components of the IEFA system separate the correct trials from the incorrect trials. In further embodiments, a tertiary component outside the IEFA system (such as a server communicatively coupled to the IEFA system) separates the correct trials from the incorrect trials.

In one embodiment, the mean RT and standard deviation (SD) for all the correct trials is calculated. In an alternate embodiment, the mean RT and SD for all incorrect trials are calculated. In a further alternate embodiment, the mean RT and SD based on both the correct trials and the incorrect trials are calculated. In one embodiment, any trials where the RT is less than or greater than 2 SD beyond the mean RT are excluded from further calculations. However, there exist alternative embodiments, where the trials where the RT is less than or greater than 2 SD beyond the mean RT bear some weight or otherwise effect the non-excluded trials or final calculation in some way. There exist alternative embodiments where the median RT is calculated. In further alternative embodiments, trials where the RT is less than or greater than 2 SD beyond the median RT are excluded from further calculations. In further alternate embodiments, the IEFA system calculates the range, mode, or other data characteristics of the correct trials and/or incorrect trials.

In one embodiment, data from the excluded trials are split into two groups: (1) trials in which the target stimulus 318 was presented in the location previously occupied by the eyes of the displayed human face and (2) trials in which the target stimulus 318 was presented in the location previously occupied by another feature on the displayed human face (such as the mouth). In an embodiment, after preprocessing the data, all calculations and evaluations are performed on each of the two groups separately and are compared.

In an embodiment, an average response time (ART) is calculated for all correct trials for each spatial location group. In one embodiment, the ART is defined as the sum of RTs in a group divided by the total number of trials of the same group. As a non-limiting example, a user may have an ART of 800 milliseconds for trials in which the target stimulus 318 was presented in the spatial location of the displayed human's eye and an ART of 700 milliseconds for trials in which the target stimulus 318 was presented in the spatial location of the displayed human's chin.

In an embodiment, a user's gaze impairment or eye contact aversion bias is evaluated by comparing the ART for each spatial location. In one embodiment, the user's gaze or eye contact aversion bias is calculated as the difference between the ART for one spatial location (such as the eyes) and the ART for another spatial location (such as the mouth). The gaze or eye contact aversion bias may be calculated during an assessment phase. The gaze or attention bias may be calculated before and after the training phase to determine any training effects on the gaze or eye contact aversion biases.

A lower relative ART may indicate a biased gaze towards that respective spatial location on the displayed human face 322. Further, the difference in ARTs may indicate which way a user is biased and by what magnitude. As a non-limiting example, a user may have an ART of 800 milliseconds for trials in which the target stimulus 318 was presented in the spatial location of the displayed human's eye(s) (for example, the first spatial location 314) and an ART of 700 milliseconds for trials in which the target stimulus 318 was presented in the spatial location of the displayed human's chin (for example, the second spatial location 316). In such a non-limiting example, the user would have a bias away from the eyes (or towards the chin) with a magnitude of 100 milliseconds.

In an embodiment, feedback is provided to the user. Feedback may be provided to the user after each trial, mid-session, or at the end of each session. In one embodiment, the feedback includes, but is not limited to, the reaction time for each trial, the average reaction time at the end of the session, and the reaction accuracy at the end of the session.

In an embodiment, the assessment phase may include 100 to 1000 trials. However, there exist alternate embodiments where the assessment phase may include less than 100 trials or more than 1000 trials. In one embodiment, target stimuli 318 are presented in the spatial location previously occupied by the displayed human's eyes in 50% of trials and in the spatial location previously occupied by another part of the displayed human face 322 for the other 50% of trials. In an embodiment, the trials may have the target stimulus 318 over the spatial location previously occupied by the displayed human's eyes in 60% of trials and the target stimulus over the mouth in 40% of trials. In various embodiments, the assessment phase may have various ratios for how many trials contain the target stimulus positioned over the eyes as compared to the target stimulus positioned over another part of the displayed human face 322. In these various embodiments, the ratio of eye to other facial component may be weighted in the final calculation of RT, ART, and/or eye contact aversion bias.

In further embodiments, a session may contain trials that have targeted stimuli 318 over more than two spatial locations. In such embodiments, as a non-limiting example, the eyes may account for 50% of trials, while the mouth and the nose may each account for 25% of trials. In such an embodiment, the data associated with the non-eye trials may be combined, or otherwise aggregated.

There exist alternative embodiments where there are more than one target stimuli disposed on the target stimulus screen at one time. As a non-limiting example, the target stimuli screen may display two indicators, one “E” positioned where the display human's eye previously occupied, and an “F” positioned where the display human's chin previously occupied. In such an embodiment, the IEFA system may instruct the user to identify which symbols occupied which spatial position.

In an embodiment, the training phase may include presentation of the target stimulus in a spatial location corresponding to the displayed human face. For example, the target stimulus may be positioned at the first feature or any feature that the system is attempting to bring attention to. For example, the eyes may be the first feature at a first spatial location. The training phase may include a location ratio. The location ratio may be the ratio between presentation of the target stimulus at the first spatial location and presentation of the target stimulus at the second spatial location. Accordingly, a location ratio in favor of the first spatial location means that, over the course of a plurality of training trials, the target stimulus will be positioned over the first spatial location more frequently. The location ratio may be represented as a percentage or in any other suitable form. As a non-limiting example, a location ratio of at least 51% in favor of the first spatial location may train the patient to make eye contact with the first spatial location. Accordingly, the location ratio may be modified over the course of the training trials or training sessions. The second spatial location may be any location that is not the first spatial location. Effectively, as it translates to facial features, the first spatial location may be the displayed human face's eyes and the second spatial location may be, for example, the mouth, right ear, chin, or forehead. In an embodiment, the first spatial location may remain the same across all trials and/or sessions, while the second spatial location changes to various features across various trials and/or sessions. By displaying the target stimulus over the eyes, the system may train the patient to gaze towards the eyes. The first spatial location (the eyes) may be the midpoint between the eyes, both the centers of the right eye and the left eye, the center of the right eye, the center of the left eye, or may be a predetermined vicinity surrounding the eyes (for example, an oblong oval surrounding the eyes).

In an embodiment, the training phase consists of multiple sessions, ranging from 1 to 1000 trials each. In an embodiment, the number of trials per session may be variable. For example, a first session may have 10 trials and a second session may have 15 trials. The system may determine the number of trials for a particular session based on the patient's past performance, or performance during an assessment phase. As a non-limiting example, if the patient has had a large number of successful training sessions, then the following training sessions may be arranged to have fewer trials. As another non-limiting example, the types of facial stimuli that the patient correctly or incorrectly answers may influence the number of trials. In such a non-limiting example, the system may generate sessions with a larger number of trials comprising the facial stimulus that the patient found most difficult. In another embodiment, the number of trials and sessions is determined based on the user's assessed attention bias. Each trial may be assigned a trial identifier. The trial identifier may be a tag associated with one trial. For example, the trial identifier may be used by the system to track data associated with that particular trial.

In an embodiment, the IEFA system creates a treatment plan for a user based on their performance in an assessment phase. The treatment plan may comprise a particular number of sessions, length of sessions, and intervals between sessions. For example, the treatment plan may comprise the generation of two short training sessions five times per week, or it may comprise the generation of three long training sessions two times per week. Depending on a user's treatment plan, the user may access the training phase at regular time intervals (for example, anywhere from daily to once a week). The training phases' purpose may be to present a stimulus to the patient that is meant to draw the patient's visual attention to a desired feature. The desired facial feature, target stimulus/stimuli, first spatial location, and/or second spatial location may be determined from the assessment phase (for example, based on the response times and/or selection status).

In an embodiment, during the training phase, the system may record and analyze the user's selections. The training phase may capture the patient's selection and/or reaction and the algorithm may then determine the patient's gaze or eye contact aversion bias and compare it to other training phase sessions, other training phase trials, and/or the metrics determined during the assessment phase. In some embodiments, a training reaction time may be determined for each of the facial stimuli and accompanying target stimuli. The training reaction time may be calculated via the formula RT_{T}=T_{TR}−T_{TP}, wherein RT_{T} is the training response time, T_{TR} is the time of receiving the selection signal, and T_{TP} is the time when the target stimulus is generated. The T_{TR} may be a function of the input from the patient via the computer peripheral. The T_{TP} may be a function of the generation of the target stimulus over the facial stimulus.

If a patient requires continuous training on a specific facial feature, more trials may include the target stimulus over that facial feature to ensure that the patient is viewing the facial feature often. The presented images may include any type of feature (for example, facial features, such as eyes). Depending on the patient and patient's treatment, a mix of these different features (for example, eyes, nose, mouth, etc.) may be used or only one type may be used. As a non-limiting example, if a patient's facial feature gaze bias is severe, they may first start with instructions on how to seek a specific feature for a specific image (for example, an average displayed human face) before progressing to the other stimuli and image types which may be considered more advanced. Thus, at the outset of any training trial or session, the system may provide a tutorial. The tutorial content (for example, the tutorial's example displayed human face) may be a function of the user's previous performance.

In an embodiment, in addition to recording the patient's selection of stimuli at the evaluation screen, the training phase may require access to the patient's camera on their mobile phone, either front facing or back camera. However, in another embodiment, the camera may be external to the patient's mobile device or electronic device. The training phase may either capture an image of the patient's face when the stimulus screen begins or a video of the patient's face throughout the trial(s). Eye tracking software may be utilized to determine the gaze of the patient relative to the desired facial feature.

After the patient selects the location of, or the displayed stimulus (for example, at the evaluation screen), the patient's response time is analyzed, and compared to the previously determined values for the image presented. For example, the response times may be saved for each image during the assessment phase or during previous training phase trials. Accordingly, a database may be maintained with average response times to each of the presented images (for example, the displayed human faces). Thus, the patient's response time may be analyzed in relation to their previous response times to the same image or to the response times of other user's to the same image. Similarly, the patient's average response time for the training phase may be evaluated in relation to the average response time determined during the assessment phase. Patients may be informed what facial feature the stimulus was presented over and/or additional information regarding the image (for example, the emotion of the displayed human face). If the patient's attention bias and/or response time did not meet or surpass a pre-determined threshold (for example, response times extracted from the assessment phase, response times determined by the system or administrator, or response times of previous training phase trials), they may be informed of the feature that they should have viewed. If the patient's response time, gaze, or stimulus selection (for example, at the evaluation screen) does not reach the threshold determined for the displayed image, they may be informed.

Before the training phase, the displayed human face, facial feature, emotion of the displayed human face, and emotion intensity, etc. for each image used in the trials is determined through testing a group of healthy individuals without negative gaze bias. These individuals' gaze bias in response to each of the stimuli may be analyzed. The response times and desired facial feature selection (for example, the selection made at the evaluation screen) may be identified by the algorithm for each image and may be agreed upon by most of the sample group. If the desired facial feature was not identified by at least 80% of the group, it may not be included. However, the threshold for inclusion may be any percentage. The response times and correctness of the facial feature selection for each image may then be used as a baseline to compare patient attention bias. In the alternative, the group of healthy individuals is recorded via smartphone camera and the software analyzes the individuals' gazes.

Depending on the patient's baseline, a few stimuli may be selected to target the facial stimuli that the patient struggles with and a few may be randomly selected from the library of validated stimuli (for example, a library of pre-loaded and/or pre-analyzed stimuli stored on the one or more computer-readable storage devices). The patient may receive feedback on the attention bias they expressed and the intensity of that bias (for example, based on the response time). In an embodiment, if the stimulus selection and/or response time do not match the desired facial feature for the stimuli, the patient receives feedback regarding the gaze they expressed and the facial feature that they were supposed to view. In other embodiments, the patient receives positive feedback for selecting the correct stimuli or stimuli location. In an embodiment, if the stimulus selection was correct but the response time is off by more than 20% (either lower or higher), the patient receives feedback on having gotten the facial feature correct but having the response time be lower/higher than expected. In other embodiments, the patient receives positive feedback for correct stimuli selection within a threshold response time.

In an embodiment, the training phase may include one or more training phase trials; each trial may include one facial stimuli (for example, a displayed human face). The system may include a computer peripheral (for example, a button, mouse, or switch), enabling the patient to inform the system when they first notice the target stimulus. The computer peripheral may transmit a selection signal to the processor, informing the system that a selection has been made (for example, that the patient has noticed/reacted to the target stimulus). In an embodiment, the selection signal may be generated by the peripheral, wherein the peripheral is the touch screen or keypad of a smart phone. As a non-limiting example, the system may include eye tracking software and a camera configured to determine the gaze of the patient. In such a non-limiting example, the system may derive the T_{TR} from the eye tracking aspect.

In a first level of training phase difficulty, the system may present a target stimulus over a facial stimulus and record the reaction of the patient. In a second level of training phase difficulty, the system may present a target stimulus over a facial stimulus, and the patient may be instructed to determine the location of the target stimulus. In a third level of training phase difficulty, the system may present one of at least two target stimuli over a facial stimulus, and the patient may be prompted to state which of the target stimuli were presented.

In an embodiment, in the first level of training phase difficulty, the target stimulus may be presented in one of two spatial locations (the first or second spatial location). The first spatial location may correlate to a first feature. The first feature may be favorable, for example, the eyes of the displayed human face. The second feature may be unfavorable, for example, the mouth of the displayed human face. The favorability of the features may be a function of whether the feature is a focal point for those with healthy attention biases (for example, determined during clinical trials).

In any of the levels of difficulty, the training phase may include a location ratio. The location ratio may be the ratio between presentation of the target stimulus at the first spatial location and presentation of the target stimulus at the second spatial location. Accordingly, a location ratio in favor of the first spatial location may indicate that, over the course of a plurality of training trials, the target stimulus will be positioned over the first spatial location more frequently. The location ratio may be represented as a percentage or in any other suitable form. As a non-limiting example, a location ratio of at least 51% in favor of the first spatial location may train the patient to make eye contact with the first spatial location. Accordingly, the location ratio may be modified over the course of the training trials or training sessions. Moreover, in another embodiment, the second spatial location may be any location that is not the first spatial location. Effectively, as it translates to facial features, the first spatial location may be the displayed human face's eyes and the second spatial location may be, for example, the mouth, right ear, chin, or forehead. In an embodiment, the first spatial location may remain the same across all trials and/or sessions, while the second spatial location changes to various features across various trials and/or sessions.

The response time per training trial, average response time per training session, selection status per training trial, and/or average selection status per training session may be determined over the course of the training phase. The location ratio may be a function of any of the aforementioned metrics determined during trials. For example, if the response time increases or does not change across a number of trials, the location ratio may increase in favorability to the first spatial location. As another example, the location ratio may be determined based on the patient's performance in an assessment phase.

In an embodiment, before beginning the assessment phase or training phase, the IEFA system presents the user with an introductory screen that enables the user to choose which phase to enter. In an embodiment, the IEFA system either does not give the user an opportunity to choose or a third party makes the determination. In one embodiment, the introductory screen or starting screen 302 presents the user with an outline for the steps in each phase, the anticipated duration of the session, and other important characteristics of the session.

In one embodiment, the memory may contain a list or spreadsheet of health conditions commonly associated with impaired eye functionality. In such an embodiment, the list or spreadsheet may also contain RTs, ARTS, and/or attention biases that are associated with particular health conditions. After completing the assessment phase, training phase, or in the middle of a session, the IEFA system may alert the user that their eye functionality is indicative of a particular health condition. In further embodiments, if the user has a high likelihood of having a severe or dangerous health condition, the IEFA system may alert emergency services.

In an embodiment, the IEFA system may also take into account other characteristics of the displayed human face 322, such as emotion, age, race, or age. In such an embodiment, the IEFA system may correlate the respective trial's RT, ART, and/or attention bias to the characteristic on the respective trial's displayed human face 322. As a non-limiting example, the IEFA system may compile a table of the attention biases of the user for the various emotions of the displayed human face 322. Further, in this non-limiting example, the IEFA system may determine that the user is more likely to be visually biased towards the eyes of a disappointed face versus the eyes of an angry face. In an embodiment, the IEFA system may include a list of mental conditions that manifest as an inability to recognize emotions. In such an embodiment, the IEFA system may alert the user or a third party (such as a physician) to a likelihood of a particular mental condition based on the user's attention bias as correlated to particular emotions.

In an embodiment, the IEFA system utilizes eye-tracking software. In such an embodiment, the IEFA system comprises a camera or a sensor that can identify and track eye movement of a user. In such an embodiment, the IEFA system may track whether a user is focusing on the eyes of the displayed human face 322 or another part of the displayed human face 322. In an embodiment, the IEFA system may utilize data collected with the aid of eye-tracking software to calculate RT. In another embodiment, the IEFA system calculates RT with the aid of eye-tracking software and also using the methods previously described. In such an embodiment, the IEFA system may calculate the difference between the two final sets of calculations (for example, the calculations determined after the training phase and the assessment phase). Further, in such an embodiment, the IEFA system may determine the likelihood that the user may answer the prompt on the evaluation screen 310 incorrectly.

In another embodiment, utilizing eye-tracking software, the IEFA system may progress through the screens without express confirmation from the user. In such an embodiment, as a non-limiting example, instead of waiting for a user to actively select one of the selectable target stimuli 320 on the evaluation screen 310, the eye-tracking software may record a selection when the user focuses on one of the prompted stimuli. In alternate embodiments, not all of the screens of the IEFA system have set durations, instead the system moves to the next screen when the user focuses on a particular point or target stimuli.

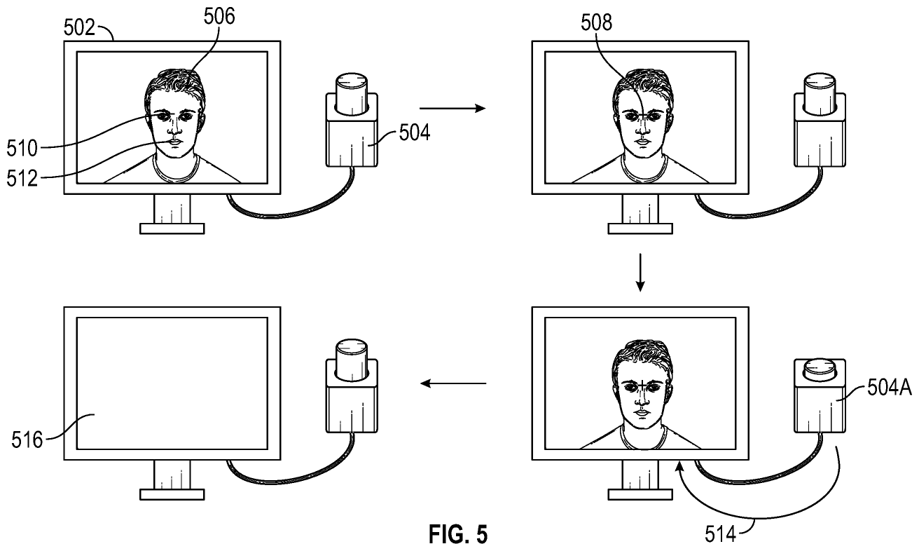

FIG. 5 is an illustration of an embodiment of the training phase. The system may include a device 502 configured to display a facial stimulus (for example, a displayed human face 506). The device 502 may be in communication with a peripheral 504 (for example, a button, mouse, keyboard, touchscreen, etc.). The displayed human face 506 may include a first spatial location 510 corresponding to a first feature (for example, the eyes) and a second spatial location 512 corresponding to a second feature (for example, the mouth). The system may generate a target stimulus 508 at the first spatial location 510 or second spatial location 512. The stimulated peripheral 504A (for example, a depressed mouse button, pressed keyboard key, or touchscreen touch) may transmit a selection signal 514 to the device 502. Once a selection signal 514 is received, the device 502 may present a blank screen 516, before presenting another displayed human face 506.

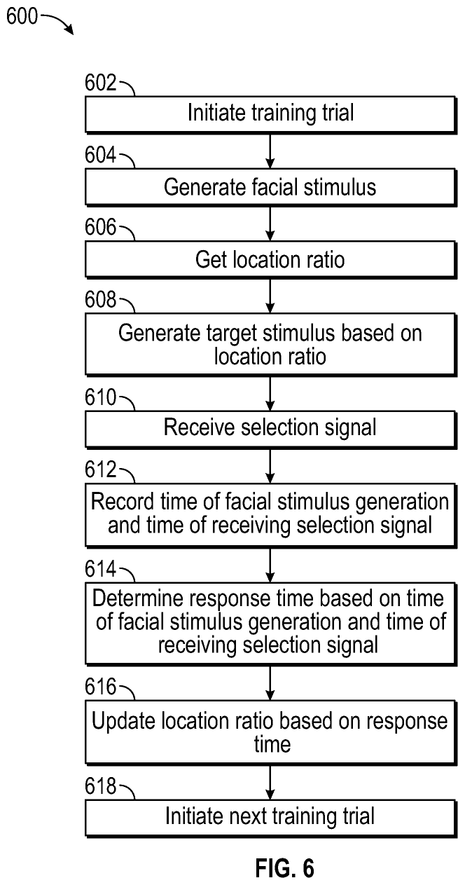

Referring to FIG. 6, the training phase may include a training phase method 600 where in step 602 a training trial is initiated. In step 604 of the method 600, a facial stimulus may be generated. In steps 606 and 608, a location ratio may be retrieved and a target stimulus may be generated based on the location ratio, respectively. In step 610, a selection signal may be received (for example, from a user interacting with a computer peripheral). In step 612, the time of the facial stimulus generation and the time of receiving the selection signal may be recorded. These times may be used in step 614 to determine the response time. In step 616, the location ratio may be updated as a function of the response time determined in step 614. In step 618, the next training trial may begin.

The invention of the present disclosure may be a computer system for assessing an eye contact aversion bias in a remote computing environment comprising one or more processors, one or more computer-readable memories, one or more displays, and one or more computer-readable storage devices, and program instructions stored on at least one of the one or more computer-readable storage devices for execution by at least one of the one or more processors via at least one of the one or more computer-readable memories. In an embodiment, the stored program instructions may comprise initiating a first predetermined number of trials, wherein a trial comprises generating, via the one or more displays, a displayed human face having a first spatial location corresponding to a first feature and a second spatial location corresponding to a second feature, wherein the first feature is the eyes of the displayed human face, and the second feature is not the eyes of the displayed human face; generating, via the one or more displays, a target stimulus, wherein the target stimulus is located at the first or the second spatial location, and receiving, via a peripheral, a selection signal. The stored program instructions may further comprise calculating, via the one or more processors, a response time via the formula RT=T_{R}−T_{P}, wherein RT is the response time, T_{R} is the time of receiving the selection signal, and T_{P} is the time when the target stimulus is generated. Further, the stored program instructions may include calculating, via the one or more processors, a first average response time for the one or more assessment trials having the target stimulus on the first spatial location; calculating, via the one or more processors, a second average response time for the one or more assessment trials having the target stimulus on the second spatial location; and calculating, via the one or more processors, the eye contact aversion bias by determining the difference between the first average response time and the second average response time.

In an embodiment, the stored program instructions further comprise determining, via the one or more processors, one or more health conditions based on the eye contact aversion bias. The stored program instructions may further comprise initiating a second predetermined number of trials, wherein the location of the target stimulus is based on a location ratio at least 51% in favor of the first spatial location. In an embodiment, the displayed human face correlates to one of a plurality of emotions. The stored program instructions may further comprise recording, via the one or more computer-readable memories, the one of the plurality of emotions; and determining, via the one or more processors, based on the response time and the plurality of emotions, whether the eye contact aversion bias is a function of the plurality of emotions.

In an embodiment, the stored program instructions may further comprise initiating a second predetermined number of trials, wherein the location of the target stimulus is based on a location ratio at least 51% in favor of the first spatial location. Further, the location ratio may be a function of the eye contact aversion bias.

The invention of the present disclosure may be a computer system for assessing an attention bias in a remote computing environment comprising one or more processors, one or more computer-readable memories, one or more displays, and one or more computer-readable storage devices, and program instructions stored on at least one of the one or more computer-readable storage devices for execution by at least one of the one or more processors via at least one of the one or more computer-readable memories. In an embodiment the stored program instructions comprise initiating one or more training trials, where each training trial comprises generating, via the one or more screens, a displayed human face; generating, via the one or more screens, at least one target stimulus, wherein the at least one target stimulus is positioned at a first spatial location or a second spatial location, wherein the displayed human face comprises the first spatial location and the second spatial location, and wherein the position of the at least one target stimulus is based on a location ratio; receiving, via a peripheral, a selection signal. The stored program instructions may further comprise calculating, via the one or more processors, a training response time via the formula RT_{T}=T_{TR}−T_{TP}, wherein RT_{T} is the training response time, T_{TR} is the time of receiving the selection signal, and T_{TP} is the time when the target stimulus is generated. In such an embodiment, the location ratio may be at least 51% in favor of the first spatial location and/or the location ratio may be a function of at least the training response time. In an embodiment, the stored program instructions further comprise initiating one or more assessment trials; generating, via the one or more displays, a starting screen having an indicator disposed at an indicator spatial location; generating, via the one or more displays, a facial stimulus screen having the first spatial location corresponding to a first feature and the second spatial location corresponding to a second feature; generating, via the one or more displays, a blank screen for an inter-stimulus interval; generating, via the one or more displays, a target stimulus screen having the at least one target stimulus; generating, via the one or more displays, an evaluation screen; presenting, via the one or more displays, on the evaluation screen, the at least one target stimulus at the first or the second spatial location, wherein the at least one target stimulus is selectable, and wherein a selection of the at least one target stimulus creates a selected target stimulus; calculating, via the one or more processors, a response time via the formula RT=T_{R}−T_{P}, wherein RT is the response time, T_{R} is the time of the selection, and T_{P} is the time when the evaluation screen is first generated; recording, via the one or more computer-readable storage devices, for each of the one or more assessment trials, the response time, and whether the at least one target stimulus was presented on the first spatial location or the second spatial location; calculating, via the one or more processors, a first average response time for the one or more assessment trials having the at least one target stimulus on the first spatial location; calculating, via the one or more processors, a second average response time for the one or more assessment trials having the at least one target stimulus on the second spatial location; calculating, via the one or more processors, an average response time differential by determining the difference between the first average response time and the second average response time; and determining, via the one or more processors, the attention bias based on the average response time differential.

The invention of the present disclosure may be a computer implemented method for treating eye contact aversion in a remote computing environment, where the method comprises initiating a first predetermined number of trials, wherein a trial comprises generating, via one or more screens, a displayed human face; generating, via the one or more screens, a target stimulus, wherein the target stimulus is positioned at a first spatial location or a second spatial location, wherein the displayed human face comprises the first spatial location and the second spatial location, and wherein the position of the target stimulus is based on a location ratio; and receiving, via a peripheral, a selection signal. The computer implemented method may further comprise initiating a second predetermined number of trials, wherein the location ratio is at least 51% in favor of the first spatial location; calculating, via the one or more processors, a response time via the formula RT=T_{R}−T_{P}, wherein RT is the response time, T_{R} is the time of receiving the selection signal, and T_{P} is the time when the target stimulus is generated; calculating, via the one or more processors, a first average response time for the trials having the target stimulus on the first spatial location; calculating, via the one or more processors, a second average response time for the one or more assessment trials having the target stimulus on the second spatial location; and calculating, via the one or more processors, an eye contact aversion bias by determining the difference between the first average response time and the second average response time. The location ratio may be at least 51% in favor of the first spatial location, wherein the first spatial location may correspond to a first feature and the second spatial location may correspond to a second feature, and wherein the first feature is the eyes of the displayed human face. In an embodiment, the location ratio is a function of the eye contact aversion bias.

While this invention has been described in conjunction with the embodiments outlined above, many alternatives, modifications and variations will be apparent to those skilled in the art upon reading the foregoing disclosure. Accordingly, the embodiments of the invention, as set forth above, are intended to be illustrative, not limiting. Various changes may be made without departing from the spirit and scope of the invention.

What is claimed is:

1. A computer system for assessing an eye contact aversion bias in a remote computing environment comprising one or more processors, one or more computer-readable memories, one or more displays, and one or more computer-readable storage devices, and program instructions stored on at least one of the one or more computer-readable storage devices for execution by at least one of the one or more processors via at least one of the one or more computer-readable memories, the stored program instructions comprising:
initiating a first predetermined number of trials, wherein a trial comprises:
generating, via the one or more displays, a displayed human face having a first spatial location corresponding to a first feature and a second spatial location corresponding to a second feature,
wherein the first feature is the eyes of the displayed human face, and the second feature is not the eyes of the displayed human face,
generating, via the one or more displays, a target stimulus, wherein the target stimulus is presented at the first or the second spatial location, and
receiving, via a peripheral, a selection signal;
calculating, via the one or more processors, a response time via the formula RT =T_{R}−T_{P},
wherein RT is the response time, T_{R} is the time of receiving the selection signal, and T_{P} is the time when the target stimulus is generated;
calculating, via the one or more processors, a first average response time for the one or more assessment trials having the target stimulus on the first spatial location;
calculating, via the one or more processors, a second average response time for the one or more assessment trials having the target stimulus on the second spatial location; and
calculating, via the one or more processors, the eye contact aversion bias by determining the difference between the first average response time and the second average response time.

2. The computer system of claim 1, the stored program instructions further comprising:
determining, via the one or more processors, one or more health conditions based on the eye contact aversion bias.

3. The computer system of claim 2, the stored program instructions further comprising:
initiating a second predetermined number of trials, wherein the location of the target stimulus is based on a location ratio at least 51% in favor of the first spatial location.

4. The computer system of claim 1, wherein the displayed human face correlates to one of a plurality of emotions.

5. The computer system of claim 4, the stored program instructions further comprising:
recording, via the one or more computer-readable memories, the one of the plurality of emotions; and
determining, via the one or more processors, based on the response time and the plurality of emotions, whether the eye contact aversion bias is a function of the plurality of emotions.

6. The computer system of claim 1 the stored program instructions further comprising:
initiating a second predetermined number of trials,
wherein the location of the target stimulus is based on a location ratio at least 51% in favor of the first spatial location.

7. The computer system of claim 6, wherein the location ratio is a function of the eye contact aversion bias.

8. A computer implemented method for treating eye contact aversion in a remote computing environment, the method comprising:
initiating a first predetermined number of trials, wherein a trial comprises:
generating, via one or more screens, a displayed human face;
generating, via the one or more screens, a target stimulus,
wherein the target stimulus is positioned at a first spatial location or a second spatial location,
wherein the displayed human face comprises the first spatial location and the second spatial location, and
wherein the position of the target stimulus is based on a location ratio; and
receiving, via a peripheral, a selection signal.

9. The computer implemented method of claim 8 further comprising:
initiating a second predetermined number of trials, wherein the location ratio is at least 51% in favor of the first spatial location, wherein the first spatial location corresponds to a first feature and the second spatial location corresponds to a second feature, and wherein the first feature is the eyes of the displayed human face.

10. The computer implemented method of claim 8, further comprising:
initiating a second predetermined number of trials, wherein the location ratio is 51% in favor of the first spatial location;
calculating, via the one or more processors, a response time via the formula RT=T_{R}−T_{P},
wherein RT is the response time, T_{R} is the time of receiving the selection signal, and T_{P} is the time when the target stimulus is generated;
calculating, via the one or more processors, a first average response time for the trials having the target stimulus on the first spatial location;
calculating, via the one or more processors, a second average response time for the one or more assessment trials having the target stimulus on the second spatial location; and
calculating, via the one or more processors, an eye contact aversion bias by determining the difference between the first average response time and the second average response time.

11. The computer implemented method of claim 9 further comprising:
initiating a second predetermined number of trials, wherein the location ratio is 50% in favor of the first spatial location;
calculating, via the one or more processors, a response time via the formula RT=T_{R}−T_{P},
wherein RT is the response time, T_{R} is the time of receiving the selection signal, and T_{P} is the time when the target stimulus is generated;
calculating, via the one or more processors, a first average response time for the trials having the target stimulus on the first spatial location;
calculating, via the one or more processors, a second average response time for the one or more assessment trials having the target stimulus on the second spatial location; and
calculating, via the one or more processors, an eye contact aversion bias by determining the difference between the first average response time and the second average response time.

12. The computer implemented method of claim 10, wherein the location ratio is a function of the eye contact aversion bias.

13. The computer implemented method of claim 11, wherein the location ratio is a function of the eye contact aversion bias.
